(19) United States
(12) Patent Application Publication
(10) Pub. No.: US 2010/0305593 A1
(43) Pub. Date: Dec. 2, 2010

(54) SAFETY SCALPEL

(76) Inventor: John R. INZERO, Cranbury, NJ (US)

Correspondence Address: Diane Dunn McKay;PORZIO, BROMBERG & NEWMAN, P.C., Suite 201, 29 Thanet Road, Princeton, NJ, 08540, US

(21) Appl. No.: 12/685,227

(22) Filed: Jan. 11, 2010

Related U.S. Application Data
(60) Provisional application No. 61/182,399, filed on May 29, 2009.

(51) Int. Cl. A61B 17/3211

(52) U.S. Cl. 606/167

(57) ABSTRACT

The present invention provides an independent activation mechanism on both sides of a plastic sheath of a safety scalpel. Activation of either one of the mechanisms on the sides will cause the sheath to slide back along the handle neck and lock in place at the locking port thereby exposing the blade for use. The activation mechanisms are thumb activated on the side of the sheath, which results in a more natural motion as well as keeping other fingers out of the way of the unsheathed blade. After use, pushing forward with the thumb on either of the activation mechanisms on the sides of the plastic sheath will cause the sheath to slide forward and lock in place. Placement of the activation mechanisms on the sides of the sheath enable the top of the sheath to be flat and smooth which provides a comfortable surface for the practitioner.

### CROSS REFERENCE TO RELATED APPLICATION

This application claims the benefit of U.S. Provisional Patent Application No. 61/182,399, filed May 29, 2009, the entirety of which is hereby incorporated by reference into this application.

### BACKGROUND OF THE INVENTION

Medical scalpels consist of a handle and a blade that attaches to the handle. It has been the case that the blades are packed in foil, and have to be manually removed from that foil packing and then attached to the handle. The handle with the exposed blade would then be passed to and from the practitioner and the assistant before, during, and after the procedure. This handling of the scalpel with its unprotected blade increases the risk of sharps injury to the practitioner and the assistant(s). OSHA has issued regulations regarding the prevention of sharps injuries. The requirement to follow them is becoming mandatory in hospitals as well as private practices.

U.S. Pat. No. 7,207,999 describes a safety scalpel including handle configured to couple to a blade and a housing configured to couple to and enclose the blade. A blade disengaging actuator is positioned on a top of the housing. A stop member is unlocked by pushing the blade disengaging actuator in a downward direction for exposing the blade. U.S. Pat. No. 7,207,999 has the shortcoming that the blade disengaging actuator can interfere with the use of the scalpel. It is desirable to provide a safety scalpel including a housing with an activation member which does not interfere with the use of the scalpel.

### SUMMARY OF THE INVENTION

The present invention provides a unique safety scalpel that helps end users address regulatory and medical issues, as well as providing greater ease of use. The present invention provides an activation mechanism on both sides of a plastic sheath of the safety scalpel. Activation of either one of the mechanisms on the sides will cause the sheath to slide back along the handle neck and lock in place at the locking port thereby exposing the blade for use. The activation mechanisms are thumb activated on the side of the sheath, which results in a more natural motion, as well as keeping other fingers out of the way of the unsheathed blade. After use, pushing forward with the thumb on either of the activation mechanisms on the sides of the plastic sheath will cause the sheath to slide forward and lock in place, thereby sheathing the blade while keeping all other fingers out of the way. Since the activation mechanisms are on the sides of the sheath, the surface at the top of the sheath is flat, providing greater comfort and stability for the practitioner.

The invention will be more fully described by reference to the following drawings.

### BRIEF DESCRIPTION OF THE DRAWINGS

FIG. 1 is top view of the safety scalpel in a closed position of the present invention.

FIG. 2 is a left side view of the safety scalpel in a closed position of the present invention.

FIG. 3 is a right side view of the safety scalpel in a closed position of the present invention.

FIG. 4 is a left side view of the safety scalpel in an open position of the present invention.

FIG. 5 is a schematic view of the safety scalpel with a top portion of the safety sheath housing removed for illustrative purposes.

FIG. 6 is a schematic view of the safety scalpel with top and side portions of the safety sheath housing removed for illustrative purposes.

FIG. 7 is a schematic view of the safety scalpel being detached from the handle.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

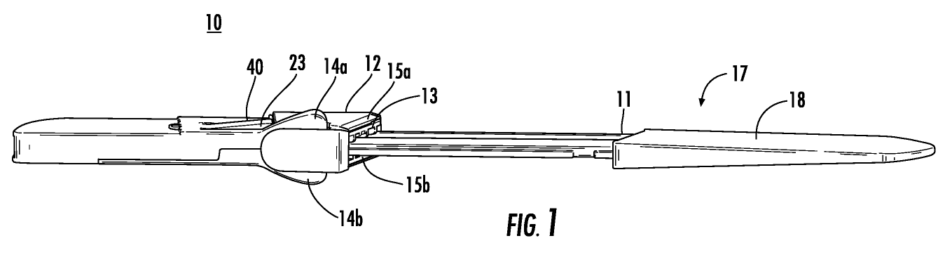

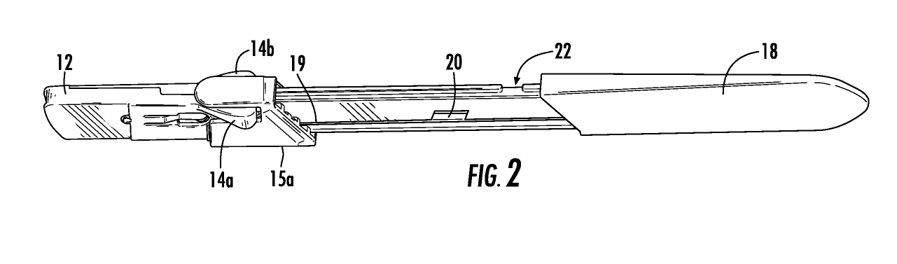

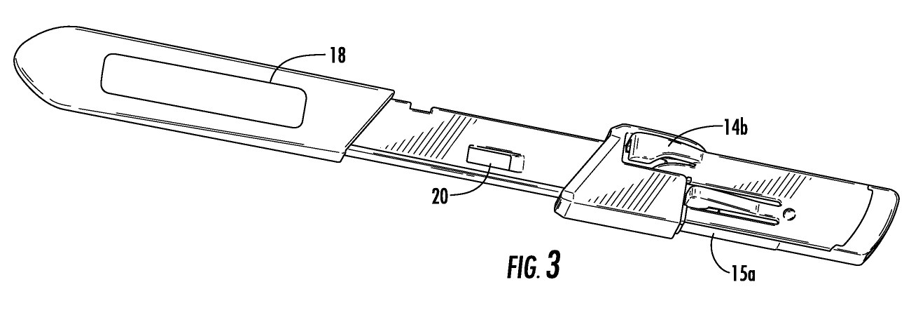

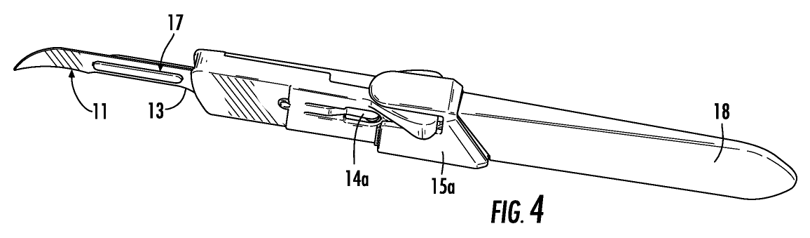

FIGS. 1-4 illustrate safety scalpel 10 in accordance with the teachings of the present invention. Blade 11 is covered by safety sheath 12 at end 13 in the closed position, as shown in FIGS. 1-3. Safety sheath 12 includes activation mechanism 14a, 14b on either side 15a, 15b of safety sheath 12. Safety sheath 12 covers end 13 of scalpel 17. Scalpel 17 includes blade 11 (covered by safety sheath 12) and handle 18. For example, blade 11 can be formed of metal and safety sheath 12 can be formed of plastic.

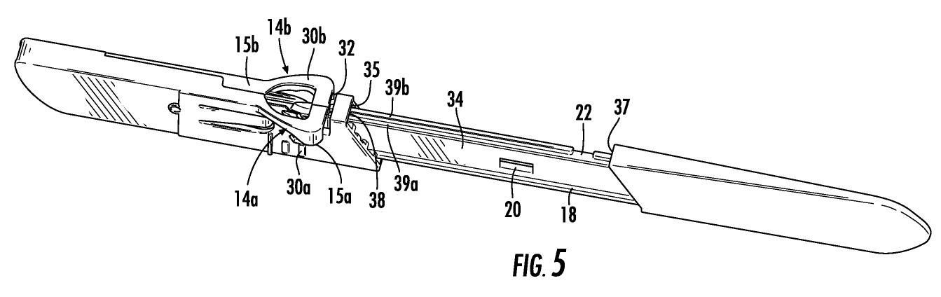

Activation mechanisms 14a, 14b each include respective flange 30a, 30b as shown in FIG. 5. Protrusion 32 extends between flanges 30a and 30b. Raised edge 34 extends along handle 18 from closed position 35 to open position 37. In closed position 35 protrusion 32 is centered adjacent end 38 of raised edge 34. In this position, raised edge 34 prevents protrusion 32 from sliding and maintains safety sheath 12 in a closed position. Activation of either of activation mechanisms 14a, 14b on sides 15a, 15b moves protrusion 32 to a respective side 39a, 39b of raised edge 34 for releasing safety sheath 12 and allowing safety sheath 12 to slide back along raised edge 34 on handle neck 20 to open position 37. If both of activation mechanisms 14a and 14b are activated protrusion 32 remains adjacent raised edge 34 and sheath 12 will not move. Release of activation mechanisms 14a, 14b at open position 37 allows protrusion 32 to lock in place at locking port 22 thereby exposing blade 11 for use. After use and while in an open position, activation of either one of activation mechanisms 14a, 14b on sides 15a, 15b releases protrusion 32 from locking port 22 to allow sheath 12 to move forward and cover blade 11 and lock back into place at end 13 at closed position 35. Activation mechanism 14a, 14b can be activated by a user's thumb. Activation mechanism 14a can be depressed for a right handed user and activation mechanism 14b can be depressed for a left handed user.

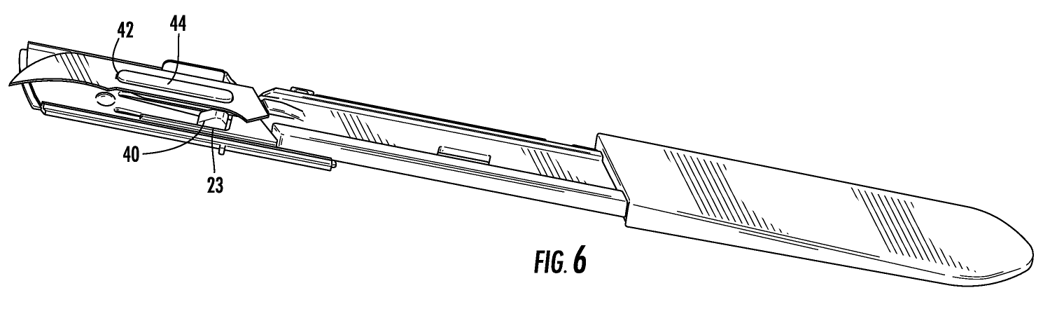

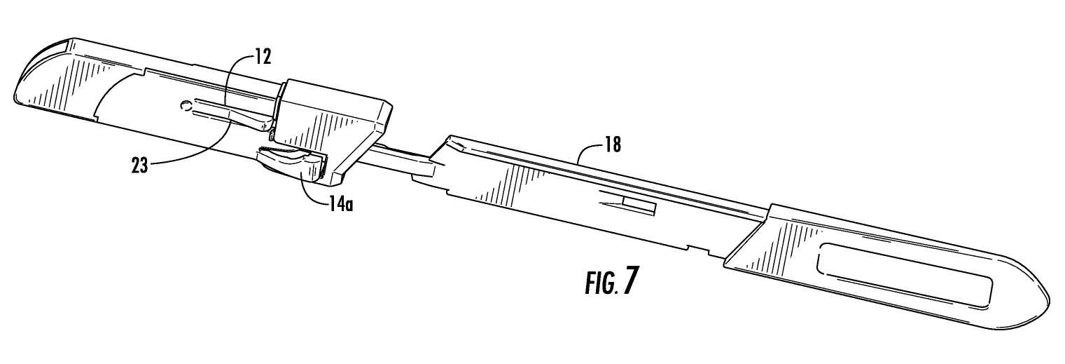

Ejection mechanism 23 can release safety sheath 12 and blade 11 inside it from handle 18, enabling safe disposal. Blade 12 covered by safety sheath 12 is removably coupled to handle 18 as shown in FIG. 6. Opening 42 in blade 12 is received over matching protrusion 44 of handle 18 thereby coupling blade 12 to handle 18. Ejection mechanism 23 includes button 40. Pushing on button 40 of ejection mechanism 23 raises blade 12 from protrusion 44 and allows safety sheath 12 to be released from handle 18 as shown in FIG. 7. Ejection mechanism 23 is on the same side as one of activation mechanism 14a or 14b. In one embodiment, ejection mechanism 23 is on the same side as activation mechanism 14a for a right handed user and on the same side as activation mechanism 14b for a left handed user.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A safety scalpel comprising:
a blade having a front cutting end;
a handle at a rear end of said blade;
a safety sheath for entirely covering said blade in a closed position of said safety sheath;
a locking port positioned on said handle; and
an independent activation mechanism positioned on either side surface of said safety sheath, wherein activation of only one of said activation mechanisms allows said safety sheath to be moved rearwardly and be locked into said locking port for exposing said blade in an open position of said safety sheath.

2. The safety scalpel of claim 1 wherein said activation mechanism includes pair of flanges and a protrusion positioned between the flanges and said handle includes a raised edge extending from the closed position to the open position wherein in a closed position said raised edge prevents said protrusion from sliding along said raised edge and in an open position said protrusion is moved to a side of said raised edge to allow said safety sheath to move along said raised edge to said open position.

3. The safety scalpel of claim 1 wherein a top of said safety sheath is substantially flat.

4. The safety scalpel of claim 1 further comprising ejecting mechanism for ejecting said blade and safety sheath from said handle said ejecting mechanism being positioned on a side of said safety scalpel.

5. The safety scalpel of claim 4 wherein said blade includes an opening received over a matching protrusion of said handle for removably coupling said handle to said blade.

6. The safety scalpel of claim 4 wherein said ejection mechanism is positioned on the same side surface of said safety sheath as said activation mechanism which is activated.

7. The safety scalpel of claim 5 wherein said ejection mechanism includes a button wherein pushing on said button raises said opening in said blade from said protrusion and allows said safety sheath and said blade to be released from said handle.

8. The safety scalpel of claim 1 wherein said handle is formed of metal.

9. The safety scalpel of claim 1 wherein said safety sheath is formed of plastic.

10. The safety scalpel of claim 1 wherein said activation mechanism is activated by a thumb of a user.

11. The safety scalpel of claim 2 wherein during activation of both of said activation mechanisms said protrusion remains adjacent said raised edge and said safety sheath does not move.

12. A safety scalpel comprising:
a blade having a front cutting end;
a handle at a rear end of said blade;
a safety sheath for entirely covering said blade in a closed position of said safety sheath;
a locking port positioned on said handle; and
an independent activation mechanism positioned on either side surface of said safety sheath, said activation mechanism includes pair of flanges and a protrusion positioned between the flanges and said handle includes a raised edge extending from the closed position to the open position wherein in a closed position said raised edge prevents said protrusion from sliding along said raised edge and in an open position said protrusion is moved to a side of said raised edge to allow said safety sheath to move along said raised edge to said open position.
wherein activation of only one of said activation mechanisms allows said safety sheath to be moved rearwardly and be locked into said locking port for exposing said blade in an open position of said safety sheath.

13. The safety scalpel of claim 12 wherein a top of said safety sheath is substantially flat.

14. The safety scalpel of claim 12 further comprising ejecting mechanism for ejecting said blade and safety sheath from said handle said ejecting mechanism being positioned on a side of said safety scalpel.

15. The safety scalpel of claim 14 wherein said blade includes an opening received over a matching protrusion of said handle for removably coupling said handle to said blade.

16. The safety scalpel of claim 15 wherein said ejection mechanism includes a button wherein pushing on said button raises said opening in said blade from said protrusion and allows said safety sheath and said blade to be released from said handle.

17. The safety scalpel of claim 14 wherein said ejection mechanism is positioned on the same side surface of said safety sheath as said activation mechanism which is activated.

18. The safety scalpel of claim 12 wherein said handle is formed of metal.

19. The safety scalpel of claim 12 wherein said safety sheath is formed of plastic.

20. The safety scalpel of claim 12 wherein during activation of both of said activation mechanisms said protrusion remains adjacent said raised edge and said safety sheath does not move.

21. The safety scalpel of claim 12 wherein said activation mechanism is activated by a thumb of a user.

22. A safety scalpel comprising:
a blade having a front cutting end;
a handle at a rear end of said blade;
a safety sheath for entirely covering said blade in a closed position of said safety sheath;
a locking port positioned on said handle; and
an independent activation mechanism positioned on either side surface of said safety sheath, wherein a top of said safety sheath is substantially flat and
activation of only one of said activation mechanisms allows said safety sheath to be moved rearwardly and be locked into said locking port for exposing said blade in an open position of said safety sheath.

23. The safety scalpel of claim 22 wherein during activation of both of said activation mechanisms said protrusion remains adjacent said raised edge and said safety sheath does not move.

24. The safety scalpel of claim 22 wherein said safety sheath is formed of plastic.

25. The safety scalpel of claim 22 wherein said activation mechanism is activated by a thumb of a user.
